(19) United States
(12) Patent Application Publication
(10) Pub. No.: US 2021/0361421 A1
(43) Pub. Date: Nov. 25, 2021

(54) REINFORCED REGENERATIVE HEART VALVES

(71) Applicant: Edwards Lifesciences Corporation, rvine, CA (US)

(72) Inventors: Ankita Bordoloi Gurunath, Irvine, CA (US); Hao Shang, Irvine, CA (US); Jingjia Han, Irvine, CA (US)

(21) Appl. No.: 17/393,624

(22) Filed: Aug. 4, 2021

Related U.S. Application Data
(63) Continuation of application No. PCT/US2020/015892, filed on Jan. 30, 2020.
(60) Provisional application No. 62/800,853, filed on Feb. 4, 2019.

(51) Int. Cl. A61F 2/24; A61L 27/58; A61L 27/36; A61L 27/38; A61L 27/54

(52) U.S. Cl. CPC A61F 2/2418; A61L 27/58; A61L 27/3625; A61L 27/3687; A61L 2430/20; A61L 27/54; A61F 2220/0075; A61F 2220/005; A61F 2250/0067; A61L 27/3834

(57) ABSTRACT

Devices and methods for reinforcing a regenerative heart valve are provided. A reinforcing element can provide structure and rigidity to withstand stresses that occur within the aortic root. In some instances, a support ring is attached to a regenerative heart valve. In some instances, a tubular wall is provided surrounding a regenerative heart valve.

### CROSS REFERENCE TO RELATED APPLICATIONS

This application is a continuation of International Patent Application No. PCT/US2020/015892, filed Jan. 30, 2020, which claims the benefit of U.S. Patent Application No. 62/800,853, filed Feb. 4, 2019, the entire disclosures all of which are incorporated by reference for all purposes.

### TECHNICAL FIELD

The application is generally directed to regenerative heart valves, and more specifically to reinforced regenerative heart valves for heart valve replacement.

### BACKGROUND

Valvular stenosis and regurgitation are a few of number of complications that may necessitate a heart valve replacement. Traditional replacement valves are constructed from various biocompatible metals, polymers and animal pericardium tissue. These valvular prostheses often have known limitations, including lifetime use of blood thinners, valve lifetime expectancy of 10 to 20 years, and/or inability to accommodate growth in children. Accordingly, a heart valve capable of growing and integrating within the site of replacement is desired.

Regenerative tissue heart valves are an intriguing solution to overcome the limitations of traditional replacement valves. Regenerative tissue heart valves are bioengineered valves produced in vitro. Because regenerative valves are live growing tissue, the valves have plasticity and remodeling capability that may allow them to integrate and grow at a site of replacement. Based on these qualities, regenerative tissue valves are a highly desirable option for procedures requiring valve replacement.

### SUMMARY OF THE INVENTION

Many embodiments are directed to devices and methods to reinforce regenerative heart valves.

In an embodiment, an implantable device for heart valve replacement includes a regenerative heart valve comprising regenerative tissue and a first ring structure adapted to be situated at the base of the heart valve to provide support for the regenerative tissue such that when the heart valve is situated at the site of replacement, the regenerative tissue can grow and integrate with native tissue while maintaining the valvular shape of the heart valve.

In another embodiment, an implantable device for heart valve replacement further includes a first tissue layer encasing the first ring structure such that the first tissue layer mitigates the first ring structure from being exposed to the native surrounding tissue when situated at the site of replacement.

In yet another embodiment, the heart valve is an aortic valve and the first ring structure provides sufficient support such that the regenerative tissue is able to grow in presence of forces that occur in the native aortic root.

In a further embodiment, the first ring structure is further adapted to expand as the heart valve annulus expands.

In still yet another embodiment, the first ring structure is segmented into at least one segment having two overlapping ends that allow expansion.

In an even further embodiment, the two overlapping ends are fastened together using a pin on a first end and a receptive guide on a second end.

In still yet an even further embodiment, the pin has a pinhead extending orthogonally from the first end and the guide has a hollowed portion configured to fit the pinhead, and wherein the guide further has a an aperture to allow the pin to move in one direction such that the two ends move in opposing directions.

In still yet an even further embodiment, the first ring structure is an overlapping coiled ring.

In still yet an even further embodiment, the first ring structure is a compressed garter spring.

In still yet an even further embodiment, the first ring structure is constructed from a biodegradable material.

In still yet an even further embodiment, the biodegradable material is selected from the group consisting of: polyglycolic acid (PGA), polylactic acid (PLA), poly-D-lactide (PDLA), polyurethane (PU), poly-4-hydroxybutyrate (P4HB), and polycaprolactone (PCL).

In still yet an even further embodiment, the biodegradable material is designed to degrade approximately in a timeframe selected from: 6, 12, 18, 24, 30 and 36 months.

In still yet an even further embodiment, the first tissue layer is adapted to capture degraded particles of the first ring structure.

In still yet an even further embodiment, the first ring structure is constructed from a metallic material.

In still yet an even further embodiment, the metallic material is selected from the group consisting of: stainless steel, cobalt-chromium alloys, titanium, and titanium alloys.

In still yet an even further embodiment, the first ring structure is attached to the base of the heart valve, and wherein the attachment is provided by sutures or an adhesive.

In still yet an even further embodiment, a second ring structure adapted to be situated on the effluent side of the heart valve to provide support for the regenerative tissue such that when the heart valve is situated at the site of replacement, the regenerative tissue can grow and integrate with native tissue while maintaining the valvular shape of the heart valve and a second tissue layer encasing the second ring structure, wherein the second tissue layer mitigates the first ring structure from being exposed to the native surrounding tissue when situated at the site of replacement.

In still yet an even further embodiment, the second ring is expandable.

In still yet an even further embodiment, the tissue sleeve is formed from pericardial tissue derived from an animal source.

In still yet an even further embodiment, the tissue sleeve is formed from autologous tissue derived from an individual to be treated.

In still yet an even further embodiment, the tissue of the regenerative heart valve is formed in vitro.

In still yet an even further embodiment, the tissue of the regenerative heart valve is formed from autologous tissue derived from an individual to be treated.

In still yet an even further embodiment, the tissue of the regenerative heart valve is grown a biodegradable scaffold.

In still yet an even further embodiment, the biodegradable scaffold is made of material selected from a group consisting of: collagen, chitosan, decellularized extracellular matrix, alginate, and fibrin.

In still yet an even further embodiment, the regenerative heart valve is trained in a bioreactor system that simulates physiological and mechanical pressures that occur in the aortic root.

In still yet an even further embodiment, the tissue of the regenerative heart valve is grown from a cell source selected from the group consisting of: mesenchymal stem cells, cardiac progenitor cells, endothelial progenitor cells, adipose tissue, vascular tissues, amniotic fluid-derived cells, and cells differentiated from pluripotent stem cells.

In still yet an even further embodiment, the cell source is mesenchymal stem cells derived from human bone marrow.

In still yet an even further embodiment, the cell source is vascular tissue derived from peripheral arteries or umbilical veins.

In still yet an even further embodiment, the tissue of the regenerative heart valve incorporates bioactive molecules.

In still yet an even further embodiment, the biomolecules promote regeneration and differentiation.

In still yet an even further embodiment, the biomolecules are selected from the group consisting of: vascular endothelial growth factor (VEGF), basic fibroblast growth factor (bFGF), transforming growth factor-β (TGF-β), angiopoietin 1 (ANGPT1), angiopoietin 2 (ANGPT2), insulin-like growth factor 1 (IGF-1) and stromal-derived factor-1-α (SDF-1-α).

In still yet an even further embodiment, the biomolecules mitigate inflammation and immune-mediated destruction of the regenerative valve.

In an embodiment, an implantable device for supporting tissue regeneration at a heart valve includes a regenerative heart valve comprising regenerative animal tissue and a tubular wall adapted to be situated to surround the effluent side of the regenerative heart valve when implanted into an individual, the tubular is further adapted to provide rigid support for the regenerative heart valve such that when situated on the effluent side of the heart valve the regenerative tissue can grow and integrate with native tissue while maintaining the valvular shape of the heart valve.

In another embodiment, the heart valve is an aortic valve and the tubular wall provides sufficient support such that the regenerative tissue is able to grow in presence of forces that occur in the native aortic root.

In yet another embodiment, the internal face of the tubular wall is engineered to promote regeneration of the regenerative heart valve and the native surrounding tissue.

In a further embodiment, the internal face of the tubular wall has a contour pattern that includes a set of ridges or furrows that are spaced such that regenerative cells are able to align and pattern to assist in formation of an endothelium-like tissue layer.

In still yet another embodiment, the set of ridges or furrows are offset at a distance that is greater than the average size of a cell associated with pannus formation.

In still yet an even further embodiment, the internal face is coated or impregnated with bioactive molecules.

In still yet an even further embodiment, the bioactive molecules promote vascular regeneration and differentiation.

In still yet an even further embodiment, the bioactive molecules attracts native endothelial progenitors.

In still yet an even further embodiment, the bioactive molecules are selected from the group consisting of: vascular endothelial growth factor (VEGF), basic fibroblast growth factor (bFGF), transforming growth factor-β (TGF-β), angiopoietin 1 (ANGPT1), angiopoietin 2 (ANGPT2), insulin-like growth factor 1 (IGF-1) and stromal-derived factor-1-α (SDF-1-α).

In still yet an even further embodiment, the biomolecules mitigate inflammation and immune-mediated destruction of the regenerative valve.

In still yet an even further embodiment, biological cells are integrated within or coated onto the internal face.

In still yet an even further embodiment, the cells are derived from an autologous source.

In still yet an even further embodiment, the cells are derived from a source selected from: mesenchymal stem cells, cardiac progenitor cells, endothelial progenitor cells, adipose tissue, vascular tissues, amniotic fluid-derived cells, and cells differentiated from pluripotent stem cells.

In still yet an even further embodiment, the cell source is mesenchymal stem cells derived from human bone marrow.

In still yet an even further embodiment, the cell source is vascular tissue derived from peripheral arteries or umbilical veins.

In still yet an even further embodiment, the tubular wall is attached the regenerative heart valve, and wherein the attachment is provided by sutures or an adhesive.

In still yet an even further embodiment, the tubular is constructed from a biodegradable material.

In still yet an even further embodiment, the biodegradable material is selected from the group consisting of: polyglycolic acid (PGA), polylactic acid (PLA), poly-D-lactide (PDLA), polyurethane (PU), poly-4-hydroxybutyrate (P4HB), and polycaprolactone (PCL).

In still yet an even further embodiment, the biodegradable material is designed to degrade approximately in a timeframe selected from: 6, 12, 18, 24, 30 and 36 months.

In still yet an even further embodiment, the tissue of the regenerative heart valve is formed in vitro.

In still yet an even further embodiment, the tissue of the regenerative heart valve is formed from autologous tissue derived from an individual to be treated.

In still yet an even further embodiment, the tissue of the regenerative heart valve is grown a biodegradable scaffold.

In still yet an even further embodiment, the biodegradable scaffold is made of material selected from the group consisting of: collagen, chitosan, decellularized extracellular matrix, alginate, and fibrin.

In still yet an even further embodiment, the regenerative heart valve is trained in a bioreactor system that simulates physiological and mechanical pressures that occur in the aortic root.

In still yet an even further embodiment, the tissue of the regenerative heart valve is grown from a cell source selected from the group consisting of: mesenchymal stem cells, cardiac progenitor cells, endothelial progenitor cells, adipose tissue, vascular tissues, amniotic fluid-derived cells, and cells differentiated from pluripotent stem cells.

In still yet an even further embodiment, the cell source is mesenchymal stem cells derived from human bone marrow.

In still yet an even further embodiment, the cell source is vascular tissue derived from peripheral arteries or umbilical veins.

In still yet an even further embodiment, the tissue of the regenerative heart valve incorporates bioactive molecules.

In still yet an even further embodiment, the biomolecules promote regeneration and differentiation.

In still yet an even further embodiment, the biomolecules are selected from the group consisting of: vascular endothelial growth factor (VEGF), basic fibroblast growth factor (bFGF), transforming growth factor-β (TGF-β), angiopoietin 1 (ANGPT1), angiopoietin 2 (ANGPT2), insulin-like growth factor 1 (IGF-1) and stromal-derived factor-1-α (SDF-1-α).

In still yet an even further embodiment, the biomolecules mitigate inflammation and immune-mediated destruction of the regenerative valve.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures, which are presented as exemplary embodiments of the invention and should not be construed as a complete recitation of the scope of the invention.

FIG. 1A provides a perspective view illustration of an embodiment of regenerative heart valve with a support ring.

FIG. 1B provides an elevation view illustration of an embodiment of regenerative heart valve with a support ring.

FIG. 2A provides an elevation view illustration of an embodiment of regenerative heart valve with a support ring and tissue sleeve.

FIG. 2B provides a cross-sectional view illustration of an embodiment of regenerative heart valve with a support ring and tissue sleeve.

FIG. 3 provides a perspective view illustration of an embodiment of regenerative heart valve with multiple support rings.

FIG. 4 provides a top view illustration of an embodiment of a segmented ring.

FIG. 5 provides an elevation view illustration of an embodiment of a joint between two ends of a segmented ring.

FIG. 6A provides an exploded perspective view illustration of an embodiment of a joint between two ends fastened using a pin and guide for use with a segmented ring.

FIG. 6B provides a top view illustration of an embodiment of an end having a guide for use with a segmented ring.

FIG. 7 provides a top view illustration of an embodiment of a coiled ring.

FIG. 8 provides a top view illustration of an embodiment of a garter spring ring.

FIG. 9A provides a perspective view illustration of an embodiment of a regenerative heart valve with a surrounding support wall.

FIG. 9B provides a cut-out perspective view illustration of an embodiment of a regenerative heart valve with a surrounding support wall.

### DETAILED DESCRIPTION

Turning now to the drawings, devices and methods to provide reinforced support to regenerative heart valves are described, in accordance with various embodiments of the invention. Several embodiments are directed towards reinforcing elements to provide support to a regenerative heart valve, especially when implanted into the aortic root. A reinforcing element, in accordance with several embodiments, provides structure and rigidity to withstand stresses that occur in the aortic root, where the forces related to systole and diastole pressures are strong and repetitive. In many embodiments, a reinforcing element prevents and/or mitigates a regenerative heart valve from collapsing. In some embodiments, a reinforcing element helps a regenerative heart valve maintain shape within the aortic root after implantation.

In numerous embodiments, a reinforcing element is biodegradable. A number of synthetic biodegradable polymers can be used, in accordance with various embodiments, to construct a support ring, including (but not limited to) polyglycolic acid (PGA), polylactic acid (PLA), poly-D-lactide (PDLA), polyurethane (PU), poly-4-hydroxybutyrate (P4HB), and polycaprolactone (PCL). Several embodiments are directed towards a reinforcing element that is constructed of a biocompatible metal or metal alloy, including (but not limited to) stainless steel, cobalt-chromium alloys, titanium, and titanium alloys.

In many embodiments, a support ring is attached to the base of a regenerative heart valve to reinforce the valve. In several embodiments, a support ring is encased within a tissue sleeve, providing a barrier between the ring and native tissue when implanted. In some embodiments, a support ring is expandable.

In a number of embodiments, a tubular wall is provided surrounding a regenerative heart valve such that the wall provides structural support. In some embodiments, a surrounding wall promotes regeneration of a heart valve and/or the native luminal walls within the aortic root.

The described apparatuses, systems, and methods should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatuses can be used in conjunction with other systems, methods, and apparatus.

### Regenerative Heart Valves Reinforced with a Support Ring

Several embodiments are directed towards a support ring to reinforce a regenerative heart valve. A support ring, in accordance with several embodiments, provides structure and rigidity to withstand stresses that occur within an aortic root, where the forces related to systole and diastole pressures are strong and repetitive. In many embodiments, a support ring prevents and/or mitigates a regenerative heart valve from collapsing. In some embodiments, a support ring helps a regenerative heart valve maintain shape within the aortic root after implantation.

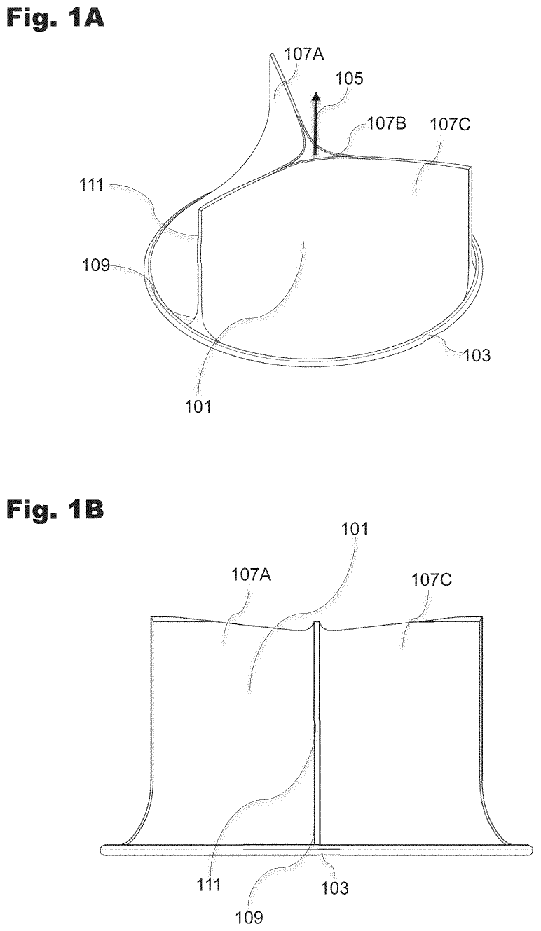

Provided in FIG. 1A is a perspective view and in FIG. 1B is an elevation view of an embodiment of a regenerative heart valve (101) having an attached ring (103) for reinforcement. In accordance with several embodiments, the heart valve (101) and attached ring (103) are to be utilized as heart valve replacement to treat heart valve disease. Numerous embodiments are directed to regenerative heart valves to replace dysfunctional aortic valves, however, it should be understood that the mitral valve, tricuspid valve, and pulmonary valve can also be replaced. Blood flow through the heart valve is depicted by arrow 105.

As can be seen in figures, the embodiment of the regenerative heart valve (101) has three leaflets (107a, 107b, and 107c) that are regenerative tissue. The leaflets are joined and/or abut at the base (109) and the side commissures (111). Typically, two or three leaflets are formulated in a regenerative heart valve, but it should be understood that number of leaflets can vary and still fall within some embodiments of the disclosure.

When replacing an aortic valve, in accordance with various embodiments, a replacement valve (101) should be situated within the aortic root such that the base (109) and attached ring (103) are located at the aortic annulus, the top of the leaflets are located at the sinotubular junction, and blood flow follows arrow 105 (e.g., from left ventricle into ascending aorta).

A number of embodiments utilize regenerative tissue to form tissue portions of a regenerative heart valve, including leaflets. In some embodiments, a regenerative heart valve is grown in vitro prior to implantation in accordance with methods as understood in the art. For more detailed discussion on regenerative heart valves, see the description described within the section labeled “Regenerative Heart Valves,” which is provided herein.

In a number of embodiments, a regenerative heart valve is to be inserted into an aortic root to replace a dysfunctional aortic valve, where the forces related to systole and diastole pressures are strong and repetitive. Because regenerative heart valves are generally composed of soft tissue and are highly plastic, they often lack sufficient rigidity to withstand strong pulsatile pressures. Thus, an implanted regenerative heart valve can collapse, causing great damage and preventing the valve from properly integrating within an aortic root. Further growth and regeneration within an aortic root can also be inhibited as host cells will not have the ability to migrate and assimilate within a regenerative valve. Accordingly, several embodiments are directed to providing a reinforcing support ring that provides structural rigidity capable of withstanding constricting and pulsatile forces associated with blood pressure in the aortic root. In many embodiments, a reinforcing support ring maintains a regenerative heart valve's shape and functionality while under stress from the blood pressure forces.

As depicted in an embodiment in FIGS. 1A and 1B, a biocompatible support ring (103) is attached to the base of a regenerative heart valve (101) at the base on the in-flow side. In several embodiments, a support ring provides rigidity and support to a regenerative heart valve. In some embodiments, a support ring is able to support a regenerative heart valve to withstand the forces within an aortic root such that the heart valve can maintain a valvular shape and continue regenerative growth post implantation. Accordingly, in some embodiments, a support ring has enough compressive strength to prevent collapse of a regenerative heart valve due to constricting forces within the aortic root Likewise, in some embodiments, a support ring has enough fatigue strength such that a regenerative heart valve is able to withstand pulsatile pressures associated with systole and diastole. As known in the art, pressures within aortic root can be approximately 120 systolic mmHg in a typical human, and can reach above 150 systolic mmHg or even 180 systolic mmHg in an individual suffering from severe hypertension. Accordingly, in various embodiments, a regenerative heart valve is able to withstand pressures of at least 100 mmHg, 110 mmHg, 120 mmHg, 130 mmHg, 140 mmHg, 150 mmHg, 160 mmHg, 170 mmHg, or 180 mmHg.

In many embodiments, a support ring is biodegradable. A number of synthetic biodegradable polymers can be used, in accordance with various embodiments, to construct a support ring, including (but not limited to) polyglycolic acid (PGA), polylactic acid (PLA), poly-D-lactide (PDLA), polyurethane (PU), poly-4-hydroxybutyrate (P4HB), and polycaprolactone (PCL). It should be understood that multiple materials can be combined to construct a support ring. In some embodiments, a support ring is degraded after implantation over a period of time, which may allow host cells to migrate into and proximate to a regenerative valve such that the host cells can support the valve after the ring is degraded. The ring will no longer be needed when the regenerative valve converts into host living tissue and adapts to the local environment, including withstanding forces within the aortic root. In various embodiments, a biodegradable support ring will degrade in a timeframe of 6 to 36 months. In some specific embodiments, a biodegradable support ring will degrade in approximately 6, 12, 18, 24, 30 or 36 months.

It should be understood that the material selected and thickness of a biodegradable support ring can be selected such that the time frame to degrade can be manipulated.

Several embodiments are directed towards a support ring that is constructed of a biocompatible metal or metal alloy, including (but not limited to) stainless steel, cobalt-chromium alloys, titanium, and titanium alloys. When a metal or metal alloy support ring is utilized, it is expected that the metal ring will remain in a regenerative valve and integrate into the host after implantation. In various embodiments, a metal or alloy support ring is durable will not corrode over time such that a host will not have issues with the ring. In some embodiments, a surface treatment and/or coating is performed on a metal or alloy support ring to resist corrosion. In some embodiments, a metal or alloy ring is adapted to be removed at some point after implantation.

In a number of embodiments, a support ring is secured to the base of a regenerative heart valve on the in-flow side. In some embodiments, a support ring is secured to the base of a regenerative heart valve using sutures. In some embodiments, sutures used to secure a support ring to the base of a regenerative heart valve are bio-absorbable. In some embodiments, a support ring is secured to the base of a regenerative heart valve using a biocompatible adhesive.

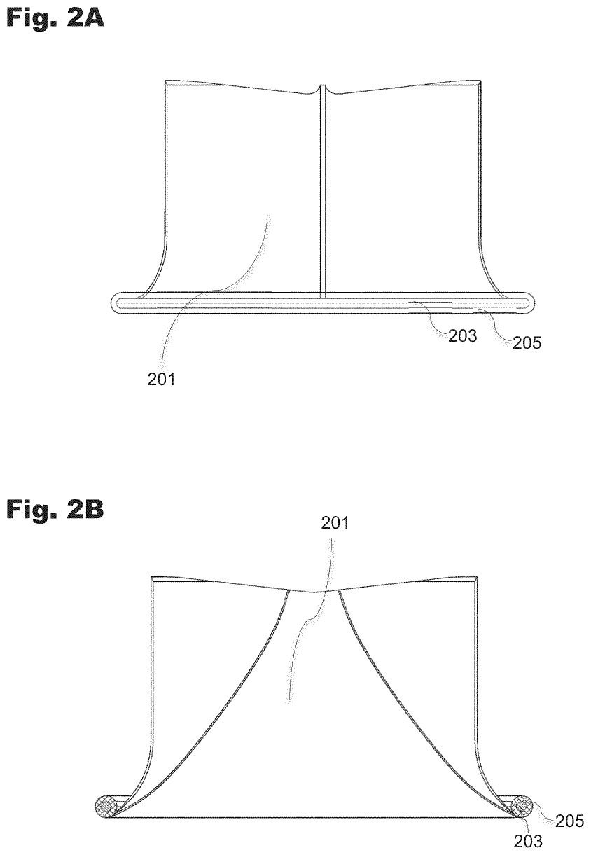

In many embodiments, a tissue sleeve encases a support ring to isolate the support ring from a host's tissue at the site of implantation. Provided in FIGS. 2A and 2B are an elevation view and cross-section view of an embodiment of a regenerative heart valve (201) with a support ring (203) attached. Encasing the support ring (203) is a tissue sleeve (205). It should be understood that any appropriate support ring constructed of any appropriate material is encased by a tissue sleeve in accordance of a number embodiments. Accordingly, in some embodiments, a tissue sleeve encases a metal or metal alloy ring. And in some embodiments, a tissue sleeve encases a biodegradable polymer.

In several embodiments, a tissue sleeve completely surrounds and encases a support ring, which may provide a number of benefits. In some embodiments, when a metal or metal alloy support ring is encased by a tissue sleeve, the tissue sleeve protects the host from direct contact with the support ring post implantation. In some embodiments when a biodegradable polymer support ring is encased by a tissue sleeve, the tissue sleeve captures degraded fragments of the support ring, preventing degraded fragments from entering into a host's circulatory system.

In accordance with many embodiments, a tissue sleeve encasing can be derived from any appropriate tissue source. In several embodiments, regenerative tissue is utilized to form a tissue sleeve, which can integrate with a host's native tissue post implantation. In some embodiments, the same regenerative tissue used to form a regenerative heart valve is used to form a tissue sleeve. In some embodiments, a tissue sleeve is formed from pericardial tissue derived from an animal source (e.g., bovine, porcine).

A tissue sleeve, in accordance with various embodiments, is grown in vitro in the presence of a support ring such that the tissue sleeve grows around the support ring to encase it. In some embodiments, a tissue sleeve is layered around a support ring and sutured to encase the support ring.

In a number of embodiments, a support ring encased in a tissue sleeve is secured to the base of a regenerative heart valve on the in-flow side. In some embodiments, a support ring is encased in a tissue sleeve secured to the base of a regenerative heart valve using sutures. In some embodiments, sutures used to secure a support ring encased in a tissue sleeve to the base of a regenerative heart valve are bio-absorbable. In some embodiments, a support ring encased in a tissue sleeve is secured to the base of a regenerative heart valve using a biocompatible adhesive.

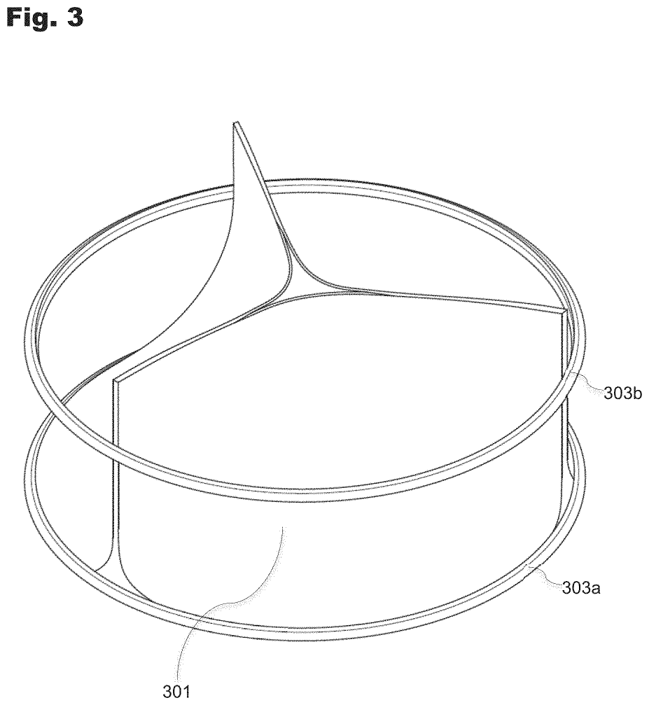

Various embodiments are also directed towards multiple support rings to provide support to a regenerative heart valve. Provided in FIG. 3 is an embodiment of a regenerative heart valve (301) having two support rings (303a and 303b). In some embodiments, a second support ring is provided along the commissures of a regenerative heart valve to further support the valve. In some embodiments, further support is provided between multiple support rings in the form of a struts or a wire mesh.

A number of embodiments are directed to methods of delivering a support ring and/or regenerative valve to the site of deployment. A method can be performed on any suitable recipient, including (but not limited to) humans, other mammals (e.g., porcine), cadavers, or anthropomorphic phantoms, as would be understood in the art. Accordingly, methods of delivery include both methods of treatment (e.g., treatment of human subjects) and methods of training and/or practice (e.g., utilizing an anthropomorphic phantom that mimics human vasculature to perform method). Methods of delivery include (but not limited to) open heart surgery and transcatheter delivery.

When a transcatheter delivery system is used, any appropriate approach may be utilized to reach the site of deployment, including (but not limited to) a transfemoral, subclavian, transapical, or transaortic approach. In several embodiments, a catheter containing a support ring and/or regenerative valve is delivered via a guidewire to the site of deployment. At the site of deployment, in accordance with many embodiments, a support ring and/or regenerative valve is released from the catheter and then expanded into form such that the support ring is at the base of a regenerative heart valve. A number of expansion mechanisms can be utilized, such as (for example) an inflatable balloon, mechanical expansion, or utilization of a self-expanding device. Particular shape designs and radiopaque regions on the frame and/or on the cover can be utilized to monitor the expansion and implementation.

Delivery and employment of a support ring and/or regenerative valve may be utilized in a variety of applications. In some embodiments, a support ring and/or regenerative device is delivered to a site for valve replacement, especially replacement of an aortic valve.

### Expandable Ring Structures

A number of embodiments are directed to support rings that are expandable. In several embodiments, a support ring, as described herein, is a ring that supports a regenerative valve from the stresses that occur within the aortic root. It is desirable in some situations that a support ring be expandable as the aortic root expands. In many embodiments, a support ring provides outward radial forces to all the ring to expand as the aortic root expands. This is especially true in heart valve replacement procedures in growing children. Accordingly, in several embodiments a support ring is expandable such that the support can expand as the regenerative valve and/or native aortic root expands.

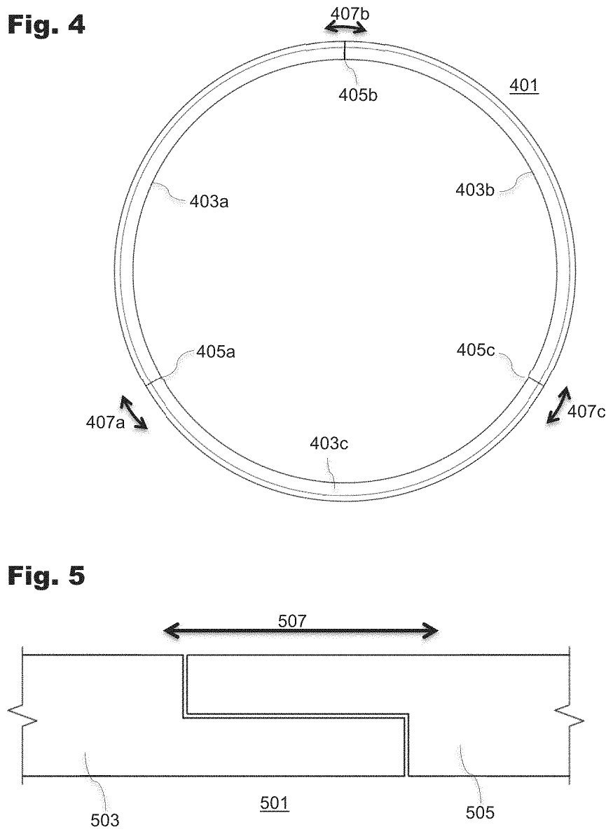

Provided in FIG. 4 is an embodiment of a segmented support ring (401) that is expandable. As shown, the segmented support ring (401) has three segments (403a, 403b, and 403c) that allow expandability at three joints (405a, 405b, and 405c). The ability to expand at the three joints is depicted by arrows (407a, 407b, and 407c). It should be understood, however, that a segmented support ring can have any appropriate number of segments and joints, but minimally must have at least 1 segment having and one joint. In various embodiments, a segmented support ring has 1, 2, 3, 4, or 5 segment(s) and joint(s).

In several embodiments, segments of a segmented support ring overlap at a joint. Provided in FIG. 5 is an elevated view an embodiment of a joint (501) of a segmented support ring in which a first end of a segment (503) and a second end of a segment (505) overlap. When the first end (503) and the second end (505) move in opposite directions as depicted in the arrow (507), the joint (501) expands and thus allowing expansion of a segmented ring. It is noted that ends (503 and 505) could be ends of a single segment or ends of two separate segments.

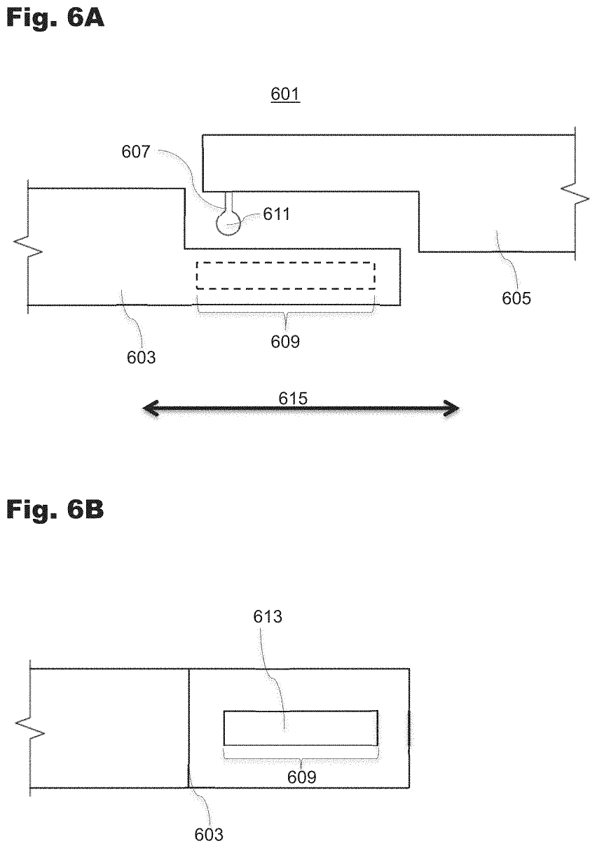

In many embodiments, overlapping segments of a segmented ring utilize a pin and guide to fasten a joint between two segment ends, but still allow expansion. Provided in FIG. 6A is an exploded view of an embodiment of a joint (601) having a first end (603) and second end (605) that utilizes a pin (607) and guide (609). Note that the guide (609) is hollowed within the first end (603). Provided in FIG. 6B is a top-down view of the first end (603) that has a guide (609) to accept the pin (607) of the second end. The pin (607) has a head (611) wider than the aperture (613) of the guide (609) to secure the ends (603 and 605) together, yet still allow the ends to move in opposite directions as depicted by the arrow (615). Expansion of the joint (601) allows the segmented ring to expand.

In numerous embodiments, a pin and guide are to be designed to such that the pin head fits within the hollowed portion of the guide but large enough that the pin head cannot pass through the aperture of the guide. Accordingly, in some embodiments, the width of the pin head is be wider than the width aperture while the width of the hollowed portion of the guide is wider than width of the pin head. Furthermore, in some embodiments, a connecting arm of the pin is to fit within the aperture of the guide such that the connecting arm can freely move in in at least one direction to allow expansion. It is noted that the shape of the pin head and the hollowed portion can vary but should be designed to work in concert such that the pin head can move freely in at least one direction within the hollowed portion. Accordingly, a pin head can be any appropriate shape, including (but not limited to) spherical, cylindrical, and cubical.

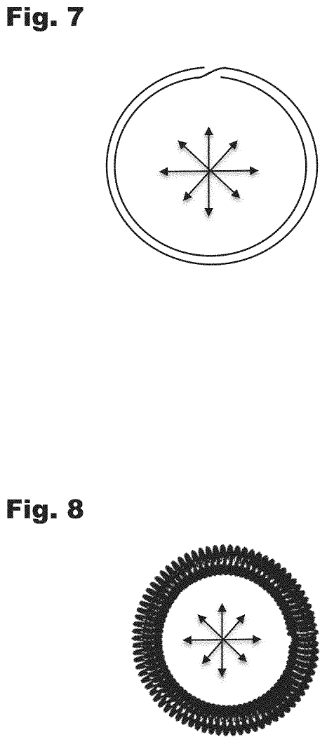

Various embodiments contemplate a number of ring-like shapes for support rings having outwardly radial forces that allow expansion while a regenerative valve expands. Provided in FIG. 7 is an embodiment of an overlapping coiled ring having outwardly radial forces. And Provided in FIG. 8 is an embodiment of a compression garter spring having outwardly radial forces. Although various drawings depict an expandable ring as segmented ring, overlapping coil ring, and a garter spring, any appropriate ring having outwardly radial forces that allow expansion can be used in accordance with a number of embodiments.

In many embodiments, an expandable support ring is biodegradable. A number of synthetic biodegradable polymers can be used, in accordance with various embodiments, to construct a support ring, including (but not limited to) polyglycolic acid (PGA), polylactic acid (PLA), poly-D-lactide (PDLA), polyurethane (PU), poly-4-hydroxybutyrate (P4HB), and polycaprolactone (PCL). It should be understood that multiple materials can be combined to construct an expandable support ring. In some embodiments, an expandable support ring is degraded after implantation over a period of time, which may allow host cells to migrate into and proximate to a regenerative valve such that the host cells can support the valve after the ring is degraded. The ring will no longer be needed when the regenerative valve converts into host living tissue and adapts to the local environment, including withstanding forces within the aortic root. In various embodiments, a biodegradable and expandable support ring will degrade in a timeframe of 6 to 36 months. In some specific embodiments, a biodegradable and expandable support ring will degrade in approximate 6, 12, 18, 24, 30 or 36 months. It should be understood that the material selected and thickness of a biodegradable and expandable support ring can be selected such that the time frame to degrade can be manipulated.

Several embodiments are directed towards an expandable support ring that is constructed of a biocompatible metal or metal alloy, including (but not limited to) stainless steel, cobalt-chromium alloys, titanium, and titanium alloys. When a metal or metal alloy expandable support ring is utilized, it is expected that the metal ring will remain in a regenerative valve and integrate into the host after implantation. In various embodiments, a metal or alloy expandable support ring is durable will not corrode over time such that a host will not have issues with the ring. In some embodiments, a surface treatment and/or coating is performed on a metal or alloy expandable support ring to resist corrosion. In some embodiments, a metal or alloy ring is adapted to be removed at some point after implantation.

In a number of embodiments, an expandable ring is secured to the base of a regenerative heart valve on the in-flow side to provide structural support. In some embodiments, an expandable support ring is secured to the base of a regenerative heart valve using sutures. In some embodiments, sutures used to secure an expandable ring to the base of a regenerative heart valve are bio-absorbable. In some embodiments, an expandable support ring is secured to the base of a regenerative heart valve using a biocompatible adhesive. In several embodiments, an expandable support ring is attached to a base of regenerative valve to provide structural support.

In numerous embodiments, a tissue sleeve completely surrounds and encases an expandable support ring, which may provide a number of benefits. In some embodiments, when a metal or metal alloy support expandable ring is encased by a tissue sleeve, the tissue sleeve protects the host from direct contact with the support ring post implantation. In some embodiments when a biodegradable polymer support ring is encased by a tissue sleeve, the tissue sleeve captures degraded fragments of the support ring, preventing degraded fragments from entering into a host's circulatory system.

### Heart Valves with Regenerative Promoting Wall

Several embodiments are directed to a regenerative heart valve having a surrounding wall. In many embodiments, a surrounding wall provides structural rigidity such that it provides structural support to a regenerative heart valve so that it can withstand stresses that occur within the aortic root. In a number of embodiments, a surrounding wall promotes regeneration of a regenerative heart valve by supplying regenerative factors that can promote host cells to migrate and convert within an implanted valve.

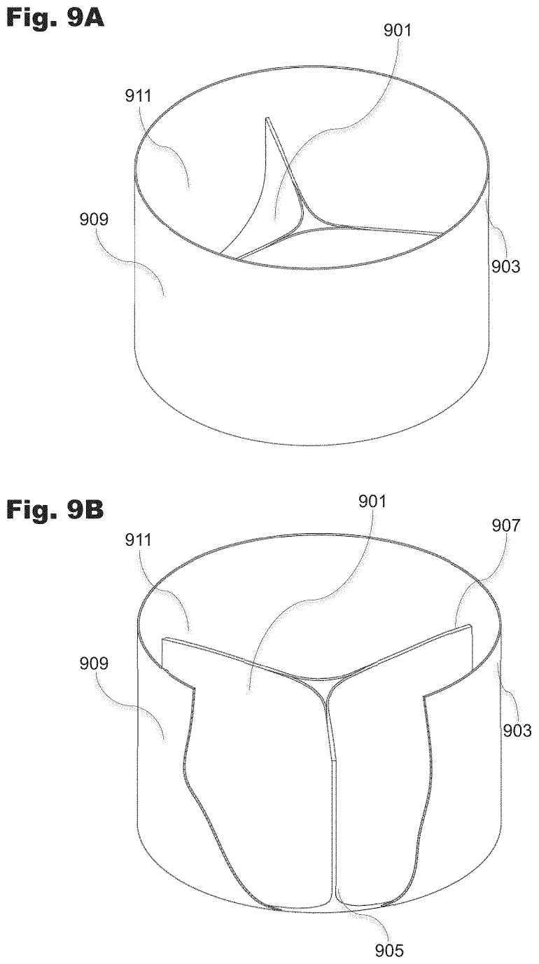

Provided in FIG. 9A is a perspective view and provided in in FIG. 9B is a perspective view with a cut-out window of an embodiment of a regenerative heart valve (901) having a surrounding wall (903). The surrounding wall (903) extends from the base area (905) of the valve to near the top or beyond the top of the leaflets (907).

In a number of embodiments, a regenerative heart valve with surrounding wall is to be inserted into an aortic root to replace a dysfunctional aortic valve. An outer face (909) of the supporting wall (903) is designed such that it contours to the native luminal surface in the aortic root. An inner face (911) of the supporting wall can be etched to form furrows and/or coated with molecules to promote cellular integration within and regeneration of the heart valve (901).

In various embodiments, a surrounding support wall provides structural support to regenerative valves within the aortic root, where the forces related to systole and diastole pressures are extremely strong and repetitive. Because regenerative heart valves are generally composed of soft tissue and are highly plastic, they lack sufficient rigidity to withstand strong pulsatile pressures. Thus, a newly implanted regenerative heart valve can be forced to collapse, causing great damage and preventing the valve from properly integrating the aortic root. Further growth and regeneration within the aortic root can also be inhibited as host cells will not have the ability to migrate and assimilate within the regenerative valve. Accordingly, several embodiments are directed to providing a reinforcing wall that provides structural rigidity capable of withstanding the constricting and pulsatile forces associated with blood pressure in the aortic root. In many embodiments, a reinforcing wall maintains a regenerative heart valve's shape and functionality while under stress from the blood pressure forces.

In some embodiments, a surrounding wall is attached to a regenerative heart valve. In some embodiments, a surrounding wall is attached at the base of a regenerative heart valve. In some embodiments, a surrounding wall is unattached to a regenerative heart valve but remains within proximity to the valve when implanted such that it is surrounding the valve.

In several embodiments, a surrounding wall provides rigidity and support to a regenerative heart valve. In some embodiments, a surrounding wall is able to support a regenerative heart valve to withstand the forces within an aortic such that the heart valve can maintain a valvular shape and continue regenerative growth post implantation. Accordingly, in some embodiments, a surrounding wall has enough compressive strength to prevent collapse of a regenerative heart valve due to constricting forces within the aortic root Likewise, in some embodiments, a surrounding wall has enough fatigue strength such that a regenerative heart valve is able to withstand pulsatile pressures associated with systole and diastole. As known in the art, pressures within aortic root can be approximately 120 systolic mmHg in a typical human, and can reach above 150 systolic mmHg or even 180 systolic mmHg in an individual suffering from severe hypertension. Accordingly, in various embodiments, a regenerative heart valve is able to withstand pressures of at least 100 mmHg, 110 mmHg, 120 mmHg, 130 mmHg, 140 mmHg, 150 mmHg, 160 mmHg, 170 mmHg, or 180 mmHg.

In many embodiments, a surrounding wall is biodegradable. A number of synthetic biodegradable polymers can be used, in accordance with various embodiments, to construct a surrounding wall, including (but not limited to) polyglycolic acid (PGA), polylactic acid (PLA), poly-D-lactide (PDLA), polyurethane (PU), poly-4-hydroxybutyrate (P4HB), and polycaprolactone (PCL). It should be understood that multiple materials can be combined to construct a surrounding wall. In some embodiments, a surrounding wall is degraded after implantation over a period of time, which may allow host cells to migrate into and proximate to the wall such that the host cells can strengthen a native aortic root wall after the implanted wall is degraded. The surrounding wall will no longer be needed when the regenerative valve converts into host living tissue and adapts to the local environment, including withstanding forces within the aortic root. In various embodiments, a biodegradable surrounding wall will degrade in a timeframe of 6 to 36 months. In some specific embodiments, a biodegradable surrounding wall will degrade in approximate 6, 12, 18, 24, 30 or 36 months. It should be understood that the material selected and thickness of a biodegradable surrounding wall can be selected such that the time frame to degrade can be manipulated.

A number of embodiments are direct to engineering the internal face of a surrounding wall to promote regeneration of a regenerative heart valve and native aortic root. In some embodiments, a surrounding wall is contoured with a micropattern on the internal face such that it promotes formation of an endothelium-like tissue layer. In some embodiments, a surrounding wall is coated and/or impregnated on the internal face with bioactive molecules to promote regeneration. In some embodiments, micropatterning and/or use of bioactive molecules prevent improper pannus formation, which can result in destructive scar tissue at the site of implantation.

In accordance with several embodiments, the internal face of a surrounding wall is contoured with a set of furrows and/or ridges to promote endothelialization and mitigate pannus formation. Methods to micropattern a surface are known in the art, such as methods described in the U.S. Patent Application Publication No. 2015/0100118 of J. A. Benton entitled “Method for Directing Cellular Migration Patterns on a Biological Tissue,” the disclosure of which is herein incorporated by reference. It is noted that polymeric surfaces, such as the internal face of a surrounding wall, can be micropatterned in a similar manner to biological tissue.

In several embodiments, micropattern includes a set of furrows and/or ridges on a surface that both dimension and offset at a distance that is greater than the average size of a fibroblast or other cell associated with pannus formation. Fibroblasts are believed to have a size in the range of 20 to 40 microns and more typically from 10 to 20 microns. Accordingly, in some embodiments, adjacent parallel furrows are offset at a distance of at least 10 microns, at least 20 microns, at least 30 microns or at least 40 microns. And in some embodiments, each individual furrow has width and/or depth of at least 10 microns, at least 20 microns, at least 30 microns or at least 40 microns. In some embodiments, parallel furrows are curved. In some embodiments, a grid pattern of intersecting parallel furrows are employed.

In many embodiments, the internal face of a surrounding wall is coated and/or impregnated with bioactive molecules to promote regeneration and differentiation within the native aortic root. Accordingly, extracellular growth factors, cytokines and/or ligands can be provided to stimulate regenerative growth and vascular differentiation. In some embodiments, factors that to be provided include (but are not limited to) vascular endothelial growth factor (VEGF), basic fibroblast growth factor (bFGF), transforming growth factor-β (TGF-β), angiopoietin 1 (ANGPT1), angiopoietin 2 (ANGPT2), insulin-like growth factor 1 (IGF-1) and stromal-derived factor-1-α (SDF-1-α). In a number of embodiments, anti-inflammatory factors are provided with regenerative tissue to mitigate inflammation and immune-mediated destruction of a regenerative valve. In some embodiments, anti-inflammatory factors to be provided include (but not limited to) curcumin and flavonoids.

In a number of embodiments, various biological cells are integrated within or coated onto the internal face of a surrounding wall that help promote regeneration and differentiation with the native aortic root. A number of cell sources can be utilized. In various embodiments, cells sources include (but are not limited to) mesenchymal stem cells (e.g., derived from bone marrow), cardiac progenitor cells, endothelial progenitor cells, adipose tissue, vascular tissues, amniotic fluid-derived cells, and cells differentiated from pluripotent stem cells. In some embodiments, vascular tissue is derived from peripheral arteries and/or umbilical veins, which can be used to isolate endothelial cells and myofibroblasts for regenerative tissue formulation. In some embodiments, pluripotent stem cells are induced into a pluripotent state from a mature cell (e.g., fibroblasts). In several embodiments, cells are sourced from an individual to be treated, which reduces concerns associated with allogenic sources.

A number of embodiments are directed to methods of delivering a surrounding wall and/or regenerative valve to the site of deployment. A method can be performed on any suitable recipient, including (but not limited to) humans, other mammals (e.g., porcine), cadavers, or anthropomorphic phantoms, as would be understood in the art. Accordingly, methods of delivery include both methods of treatment (e.g., treatment of human subjects) and methods of training and/or practice (e.g., utilizing an anthropomorphic phantom that mimics human vasculature to perform method). Methods of delivery include (but not limited to) open heart surgery and transcatheter delivery.

When a transcatheter delivery system is used, any appropriate approach may be utilized to reach the site of deployment, including (but not limited to) a transfemoral, subclavian, transapical, or transaortic approach. In several embodiments, a catheter containing a surrounding wall and/or regenerative valve is delivered via a guidewire to the site of deployment. At the site of deployment, in accordance with many embodiments, a wall and/or regenerative valve is released from the catheter and then expanded into form such that the wall is surrounding a regenerative heart valve. A number of expansion mechanisms can be utilized, such as (for example) an inflatable balloon, mechanical expansion, or utilization of a self-expanding device. Particular shape designs and radiopaque regions on the frame and/or on the cover can be utilized to monitor the expansion and implementation.

Delivery and employment of a surrounding wall and/or regenerative valve may be utilized in a variety of applications. In some embodiments, a surrounding wall and/or regenerative device is delivered to a site for valve replacement, especially replacement of an aortic valve.

### Regenerative Heart Valves

Several embodiments are directed toward the use of heart valves formed of regenerative, including leaflets. Regenerative tissue to be utilized in a regenerative heart valve can be any appropriate formulation of regenerative tissue as understood in the art. In various embodiments, regenerative tissue is formulated in vitro. In some embodiments, regenerative tissue is autologous (e.g., generated from tissue and or cells of the individual to be treated). In some embodiments, regenerative tissue is allogenic (e.g., generated from a source other than the individual to be treated). When allogenic tissue is be used, in accordance with some embodiments, appropriate measures to mitigate immunoreactivity and/or rejection of the tissue may be necessary.

In various embodiments, regenerative tissue is formulated such that regenerative heart valve is able to grow, adapt, and integrate within the aortic root after implantation. Growth and adaptation is especially critical for heart valve replacement in children, which may avoid the necessity of multiple valve replacement surgeries as the child grows. In some embodiments, a regenerative heart valve is formulated to resist thrombosis and pannus formation. In some embodiments, a regenerative heart valve is “trained” in bioreactor systems that simulate physiological and mechanical pressures that occur in the aortic root.

In accordance with several embodiments, regenerative tissue is formulated on a scaffold such that the tissue grows into an appropriate heart valve shape. In many embodiments, scaffolds are biodegradable such that when implanted and/or a short time after implantation, the scaffold degrades leaving behind only the regenerative tissue. A number of scaffold matrices can be used, as understood in the art. In some embodiments, a synthetic polymer is used, such as (for example) polyglycolic acid (PGA), polylactic acid (PLA), poly-D-lactide (PDLA), polyurethane (PU), poly-4-hydroxybutyrate (P4HB), and polycaprolactone (PCL). In some embodiments, a biological matrix is used, which can be formulated from a number of biomolecules including (but not limited to) collagen, fibrin, hyaluronic acid, alginate, and chitosan. In some embodiments, a decellularized extracellular matrix is used as a scaffold. It should be understood that various scaffold matrices can be combined and utilized in accordance with various embodiments.

A number of cell sources can be utilized in formulating regenerative tissue. In various embodiments, cells sources include (but are not limited to) mesenchymal stem cells (e.g., derived from bone marrow), cardiac progenitor cells, endothelial progenitor cells, adipose tissue, vascular tissues, amniotic fluid-derived cells, and cells differentiated from pluripotent stem cells (including embryonic stem cells). In some embodiments, vascular tissue is derived from peripheral arteries and/or umbilical veins, which can be used to isolate endothelial cells and myofibroblasts for regenerative tissue formulation. In some embodiments, pluripotent stem cells are induced into a pluripotent state from a mature cell (e.g., fibroblasts). In several embodiments, cells are sourced from an individual to be treated, which reduces concerns associated with allogenic sources.

In various embodiments, bioactive molecules including regenerative and differentiation factors are provided with regenerative tissue to stimulate host regeneration at the site implantation. Accordingly, extracellular growth factors, cytokines and/or ligands can be provided to stimulate regenerative growth and vascular differentiation. In some embodiments, factors that to be provided include (but are not limited to) vascular endothelial growth factor (VEGF), basic fibroblast growth factor (bFGF), transforming growth factor-β (TGF-β), angiopoietin 1 (ANGPT1), angiopoietin 2 (ANGPT2), insulin-like growth factor 1 (IGF-1) and stromal-derived factor-1-α (SDF-1-α). In a number of embodiments, anti-inflammatory factors are provided with regenerative tissue to mitigate inflammation and immune-mediated destruction of a regenerative valve. In some embodiments, anti-inflammatory factors to be provided include (but not limited to) curcumin and flavonoids.

In a number of embodiments, a regenerative heart valve is to be inserted into an aortic root to replace a dysfunctional aortic valve, where the forces related to systole and diastole pressures are extremely strong and repetitive. Because regenerative heart valves are generally composed of soft tissue and are highly plastic, they lack sufficient rigidity to withstand strong pulsatile pressures. Thus, a newly implanted regenerative heart valve can be forced to collapse, causing great damage and preventing the valve from properly integrating the aortic root. Further growth and regeneration within the aortic root can also be inhibited as host cells will not have the ability to migrate and assimilate within the regenerative valve. Accordingly, several embodiments are directed to providing reinforcing elements that provide structural rigidity capable of withstanding the constricting and pulsatile forces associated with blood pressure in the aortic root. In many embodiments, reinforcing elements maintain a regenerative heart valve's shape and functionality while under stress from the blood pressure forces.

### Doctrine of Equivalents

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. An implantable device for heart valve replacement, comprising:
a regenerative heart valve comprising regenerative tissue; and
a first ring structure adapted to be situated at the base of the heart valve to provide support for the regenerative tissue such that when the heart valve is situated at the site of replacement, the regenerative tissue can grow and integrate with native tissue while maintaining the valvular shape of the heart valve.

2. The device as in claim 1 further comprising a first tissue layer encasing the first ring structure, wherein the first tissue layer mitigates the first ring structure from being exposed to the native surrounding tissue when situated at the site of replacement.

3. The device as in claim 1, wherein the heart valve is an aortic valve and the first ring structure provides sufficient support such that the regenerative tissue is able to grow in presence of forces that occur in the native aortic root.

4. The device as in claim 1, wherein the first ring structure is further adapted to expand as the heart valve annulus expands.

5. The device as in claim 1, wherein the first ring structure is segmented into at least one segment having two overlapping ends that allow expansion.

6. The device as in claim 5, wherein the two overlapping ends are fastened together using a pin on a first end and a receptive guide on a second end.

7. The device as in claim 6, wherein the pin has a pinhead extending orthogonally from the first end and the guide has a hollowed portion configured to fit the pinhead, and wherein the guide further has a an aperture to allow the pin to move in one direction such that the two ends move in opposing directions.

8. The device as in claim 1, wherein the first ring structure is an overlapping coiled ring.

9. The device as in claim 1, wherein the first ring structure is a compressed garter spring.

10. The device as in claim 1, wherein the first ring structure is constructed from a biodegradable material.

11. The device as in claim 10, wherein the biodegradable material is selected from the group consisting of: polyglycolic acid (PGA), polylactic acid (PLA), poly-D-lactide (PDLA), polyurethane (PU), poly-4-hydroxybutyrate (P4HB), and polycaprolactone (PCL).

12. The device as in claim 10, wherein the biodegradable material is designed to degrade approximately in a timeframe selected from: 6, 12, 18, 24, 30 and 36 months.

13. The device as in claim 10, wherein the first tissue layer is adapted to capture degraded particles of the first ring structure.

14. The device as in claim 1, wherein the first ring structure is constructed from a metallic material.

15. The device as in claim 14, wherein the metallic material is selected from the group consisting of: stainless steel, cobalt-chromium alloys, titanium, and titanium alloys.

16. The device as in claim 1, wherein the first ring structure is attached to the base of the heart valve, and wherein the attachment is provided by sutures or an adhesive.

17. The device as in claim 1 further comprising:
a second ring structure adapted to be situated on the effluent side of the heart valve to provide support for the regenerative tissue such that when the heart valve is situated at the site of replacement, the regenerative tissue can grow and integrate with native tissue while maintaining the valvular shape of the heart valve; and
a second tissue layer encasing the second ring structure, wherein the second tissue layer mitigates the first ring structure from being exposed to the native surrounding tissue when situated at the site of replacement.

18. The device as in claim 17, wherein in the second ring is expandable.

19. The device as in claim 1, wherein the tissue sleeve is formed from pericardial tissue derived from an animal source.

20. The device as in claim 1, wherein the tissue sleeve is formed from autologous tissue derived from an individual to be treated.

21. The device as in claim 1, wherein the tissue of the regenerative heart valve is formed in vitro.

22. The device as in claim 1, wherein the tissue of the regenerative heart valve is formed from autologous tissue derived from an individual to be treated.

23. The device as in claim 1, wherein the tissue of the regenerative heart valve is grown a biodegradable scaffold.

24. The device as in claim 1, wherein the biodegradable scaffold is made of material selected from a group consisting of: collagen, fibrin, hyaluronic acid, alginate, decellularized extracellular matrix and chitosan.

25. The device as in claim 1, wherein the regenerative heart valve is trained in a bioreactor system that simulates physiological and mechanical pressures that occur in the aortic root.

26. The device as in claim 1, wherein the tissue of the regenerative heart valve is grown from a cell source selected from the group consisting of: mesenchymal stem cells, cardiac progenitor cells, endothelial progenitor cells, adipose tissue, vascular tissues, amniotic fluid-derived cells, and cells differentiated from pluripotent stem cells.

27. The device as in claim 26, where the cell source is mesenchymal stem cells derived from human bone marrow.

28. The device as in claim 26, where the cell source is vascular tissue derived from peripheral arteries or umbilical veins.

29. The device as in claim 1, wherein the tissue of the regenerative heart valve incorporates bioactive molecules.

30. The device as in claim 29, wherein the biomolecules promote regeneration and differentiation.

31. The device as in claim 29, wherein the biomolecules are selected from the group consisting of: vascular endothelial growth factor (VEGF), basic fibroblast growth factor (bFGF), transforming growth factor-β (TGF-β), angiopoietin 1 (ANGPT1), angiopoietin 2 (ANGPT2), insulin-like growth factor 1 (IGF-1) and stromal-derived factor-1-α (SDF-1-α).

32. The device as in claim 29, wherein the biomolecules mitigate inflammation and immune-mediated destruction of the regenerative valve.
